(19) United States
(12) Patent Application Publication
(10) Pub. No.: US 2021/0169748 A1
(43) Pub. Date: Jun. 10, 2021

(54) MEDICAL FILLER COMPOSITION

(71) Applicant: MARUCHI CO., LTD., Wonju-si (KR)

(72) Inventor: Sung-Wook JANG, Seoul (KR)

(21) Appl. No.: 16/761,363

(22) PCT Filed: Nov. 28, 2017
(86) PCT No.: PCT/KR2017/013677

§ 371 Date: May 4, 2020

(30) Foreign Application Priority Data
Nov. 9, 2017 (KR) 10-2017-0148512

(51) Int. Cl. A61K 6/86; A61K 6/84; A61K 6/76; A61K 6/60; A61K 6/19

(52) U.S. Cl. CPC A61K 6/86; A61K 6/84; A61K 6/19; A61K 6/60; A61K 6/76

(57) ABSTRACT

Disclosed is a medical filler composition, which is loaded in a space which is empty due to the removal of nerves, blood vessels, cell tissues and hard tissues therefrom and which is also biocompatible, highly bioactive and biochemically stable and has high workability and sealing ability, including a calcium supply source including at least one selected from among calcium hydroxide and calcium oxide, a silicon supply source including at least one selected from fumed silica, precipitated silica, colloidal silica and clay mineral, and 20 to 70 parts by weight of a liquid material for pasting, based on 100 parts by weight of a mixture of the calcium supply source and the silicon supply source, wherein the calcium/silicon molar ratio of calcium silicate hydrate produced by a pozzolanic reaction between the calcium supply source and the silicon supply source is 0.25 to 1.5.

### TECHNICAL FIELD

The present invention relates to a medical filler composition, and more particularly to a medical filler composition, which is loaded in a space that is empty due to the removal of nerves, blood vessels, cell tissues and hard tissues therefrom.

### BACKGROUND ART

Typically, a medical filler composition is utilized by being loaded into a space that is empty due to the removal of nerves, blood vessels, cell tissues and hard tissues therefrom for the purpose of treatment, and is currently applied in a variety of fields.

A medical filler composition is essential in the dental field, especially in the field of endodontic treatment, in which the space resulting from removing the nerves, blood vessels, and other cell tissues from the inside of the tooth is filled with the material and sealed to maintain the function of the tooth.

Useful as the medical filler composition, a mineral trioxide aggregate (MTA) is widely used in the field of endodontic treatment, and is mainly employed in root perforation repair, pulpotomy, partial pulpotomy, pulp capping, root canal filling, root-end retrofilling, and the like. Such MTA may exhibit superior sealing ability and biocompatibility, and is predominant in terms of the formation of tertiary dentin or the infiltration of inflammatory cells compared to calcium hydroxide, which is mainly applied in the dental pulp treatment of vital teeth.

In general, MTA is composed mainly of calcium silicate, calcium aluminate and gypsum, among which calcium silicate reacts with water in environments where body fluids, saliva, and other liquids are present, thereby forming calcium silicate hydrate (C—S—H) and calcium hydroxide. Here, the product obtained through the hydration of MTA is typically estimated to comprise about 75% calcium silicate hydrate (C—S—H) and about 25% calcium hydroxide.

The calcium silicate hydrate, which is the main hydration phase resulting from the MTA hydration, has an amorphous or semicrystalline structure, and thus C—S—H has an ambiguous chemical formula, and is therefore generally categorized by the calcium/silicon molar ratio.

More specifically, according to Tayler, calcium silicate hydrate is categorized into a tobermorite model when the calcium/silicon molar ratio is less than 1, and into a jennite model when such a molar ratio exceeds 1. According to Nonat, calcium silicate hydrate may be categorized into C—S—H(α) when the calcium/silicon molar ratio is less than 1, into C—S—H(β) at a molar ratio of 1˜1.5, and into C—S—H(γ) at a molar ratio of greater than 1.5.

Also, calcium silicate hydrate (C—S—H) has a variety of shapes, ranging from loose fiber crystals to irregular and twisted netted structures, and has a layer structure having a very high surface area and internal voids in a colloidal state, and occupies about 50 to 60% of the volume of the cured MTA.

On the other hand, calcium hydroxide has hexagonal plate-like crystals and occupies about 20 to 25% of the volume of the cured MTA. The amount of calcium hydroxide is associated with the kind of calcium silicate contained in MTA and the extent of hydration reaction.

For example, among the kinds of calcium silicate, tricalcium silicate produces calcium hydroxide while about 80% or more dissolves, whereas dicalcium silicate only partially dissolves, and thus the amount of produced calcium hydroxide is small. Furthermore, since a small amount of calcium silicate reacts at the early stage of the hydration reaction, the amount of produced calcium hydroxide is small.

Meanwhile, MTA is superior in sealing ability compared to conventionally used amalgam, IRM, and Super EBA, but is problematic in that it has a long curing time, is inconvenient to handle, and tends to discolor.

Also, MTA is greatly affected by the surrounding acidic environment during the hydration thereof and thus physical properties or structures thereof may deteriorate. Even after curing, when the cured MTA is exposed to the saliva or gingival crevicular fluid in the mouth, the structure thereof is drastically weakened, the fracture resistance of the tooth structure is weakened in the root canal, and abrupt narrowing of the pulp cavity in vital teeth may result.

This is because calcium hydroxide produced by the hydration reaction of the MTA reacts with the saliva or gingival crevicular fluid to produce gypsum, sodium hydroxide and magnesium hydroxide, thus increasing the volume thereof to thereby create expansion pressure. In fact, the molar volume of calcium hydroxide is 33.2 cm³, whereas the molar volume of gypsum is 74.2 cm³, which leads to a volume increase of about 2.2 times when calcium hydroxide is converted to gypsum.

Moreover, gypsum reacts again with calcium aluminate hydrate, monosulfate and tricalcium aluminate (C₃A) to produce ettringite. In this procedure, the volume is also increased, and thus expansion pressure occurs, thereby cracking the cured MTA.

Hence, with the goal of solving such problems, extensive research is ongoing into methods of using a pozzolanic reaction, which is a reaction in which silica and alumina components react with calcium hydroxide in the presence of water to form calcium silicate hydrate (C—S—H).

However, conventional techniques for producing calcium silicate hydrate (C—S—H) using a pozzolanic reaction have difficulties in producing calcium silicate hydrate (C—S—H) in vivo, and even if they are produced in vivo, shrinkage occurs during the curing process after injection into a space requiring in-vivo repair and filling, resulting in a problem of poor sealing ability.

### CITATION LIST

### Patent Literature

Korean Patent No. 10-1385237

### DISCLOSURE

### Technical Problem

Accordingly, the present invention has been made keeping in mind the problems encountered in the related art, and the present invention is intended to provide a medical filler composition, which is capable of forming calcium silicate hydrate (C—S—H) through a pozzolanic reaction after injection into an environment in which water is present, namely a space requiring in-vivo repair and filling, and does not shrink during curing and thus exhibits high sealing ability.

### Technical Solution

Therefore, the present invention provides a medical filler composition, comprising: a calcium supply source including at least one selected from among calcium hydroxide and calcium oxide, a silicon supply source including at least one selected from fumed silica, precipitated silica, colloidal silica and clay mineral, and 20 to 70 parts by weight of a liquid material for pasting, based on 100 parts by weight of a mixture comprising the calcium supply source and the silicon supply source, wherein the calcium/silicon molar ratio of calcium silicate hydrate produced by a pozzolanic reaction between the calcium supply source and the silicon supply source ranges from 0.25 to 1.5.

### Advantageous Effects

According to the present invention, a medical filler composition can produce calcium silicate hydrate (C—S—H) through a pozzolanic reaction after injection into an environment in which water is present, namely a space requiring in-vivo repair and filling. A cured medical filler composition containing the calcium silicate hydrate (C—S—H) thus produced is a neutral compound that is biocompatible and has high bioactivity and high biochemical stability, and can be effectively used for perforation repair of a portion that contacts the saliva or gingival crevicular fluid because it is not corroded by the saliva or gingival crevicular fluid.

Furthermore, the medical filler composition of the invention can exhibit high workability and sealing ability and is thus free from micro-leakage, thus obviating an additional operation for sealing and inhibiting secondary infection. Also, the medical filler composition of the invention does not overflow the root end even when injected with excessive pressure over a predetermined magnitude, and is thus excellent in terms of safety.

Moreover, the medical filler composition of the invention is in a paste form and is thus easily injected into a space requiring in-vivo repair and filling and can be effectively absorbed to the surrounding tissue even after injection.

### BRIEF DESCRIPTION OF DRAWINGS

FIG. 1 shows the results of scanning electron microscopy (SEM) of the cured medical filler composition of Preparation Example 1-1;

FIG. 2 shows the results of energy-dispersive X-ray spectroscopy (EDS) mapping of the cured medical filler composition of Preparation Example 1-1;

FIG. 3 shows the results of X-ray diffraction (XRD) of the cured medical filler composition of Preparation Example 1-1;

FIG. 4 shows the results of testing of cytotoxicity of the cured medical filler compositions of Preparation Examples 1-1 to 1-4; and

FIG. 5 shows the results of testing of cytotoxicity of the cured medical filler composition of Example 1 and conventional cured medical filler compositions.

### BEST MODE FOR CARRYING OUT THE INVENTION

Respective medical filler compositions were prepared by mixing calcium hydroxide (Ca(OH)₂), precipitated silica and dimethyl sulfoxide (DMSO) so as to produce calcium silicate hydrates (C—S—H) at calcium/silicon molar ratios of 1.5, 1.2, 0.9 and 0.7 through a pozzolanic reaction, as shown in Table 1 below. Each of the medical filler compositions was then exposed at a humidity of 100% and a temperature of 36° C. to thus complete curing thereof, thereby obtaining cured medical filler compositions.

### MODE FOR THE INVENTION

Hereinafter, embodiments and examples of the present invention will be described in detail so that those skilled in the art can easily carry out the present invention with reference to the accompanying drawings. However, the present invention may be embodied in many different forms, and is not limited to the embodiments and examples described herein. In order to clearly illustrate the present invention, parts not related to the description are omitted in the drawings.

As used herein, when any part “comprises” or “includes” any element, it means that other elements are not precluded but may be further included, unless otherwise mentioned.

As used herein, the term “about” is used in the sense of equaling or approximating a numerical value when a unique manufacturing and material tolerance is presented in context, and is used to prevent the disclosure from being misconstrued by unscrupulous infringers to mean an absolute and precise numerical value, in order to facilitate understanding of the present invention.

The present invention addresses a medical filler composition comprising a calcium supply source and a silicon supply source.

The calcium supply source preferably includes at least one selected from among calcium hydroxide (Ca(OH)₂) and calcium oxide (CaO). This is because calcium hydroxide is medically safe and calcium oxide has high reactivity.

Also, calcium hydroxide and calcium oxide are strong bases, and are thus effective at exhibiting antimicrobial activity, endotoxin neutralization and induction of hard-tissue formation immediately after the medical filler composition of the present invention is injected into a space requiring in-vivo repair and filling.

The silicon supply source is a material that causes a pozzolanic reaction with the calcium supply source in the presence of water, thus producing calcium silicate hydrate (C—S—H) resulting from the pozzolanic reaction, whereby the structure of the cured medical filler composition obtained by curing the medical filler composition of the present invention becomes denser, ultimately improving workability and sealing ability.

When the medical filler composition of the present invention contains, as a radiopaque powder, a ferroelectric material powder, the silicon supply source may function to prevent the ferroelectric material powder from aggregating due to electrical reaction.

The silicon supply source may include a natural silicon supply source or an artificial silicon supply source, and preferably includes at least one selected from among fumed silica, precipitated silica, colloidal silica and clay minerals.

Here, examples of clay minerals may include tuff, diatomite, zeolite, metakaolin, montmorillonite clay, a smectite clay mineral, and synthetic swellable clay.

The calcium supply source and the silicon supply source are preferably contained in the medical filler composition of the present invention so that the calcium/silicon molar ratio of the calcium silicate hydrate (C—S—H) resulting from the pozzolanic reaction therebetween is preferably 0.25 to 1.5, and more preferably 1.0 or less.

When calcium hydroxide and silicon dioxide (SiO₂) are contained as the calcium supply source and the silicon supply source, respectively, silicon dioxide may be used in an amount of 50 to 330 parts by weight based on 100 parts by weight of calcium hydroxide so that the calcium silicate hydrate (C—S—H) resulting from the pozzolanic reaction of calcium hydroxide and silicon dioxide has a calcium/silicon molar ratio of 0.25 to 1.5. As such, these amounts may be determined taking into consideration the calcium hydroxide molecular weight of 74.093 and the silicon dioxide molecular weight of 60.09.

The calcium silicate hydrate (C—S—H) having a calcium/silicon molar ratio of 0.25 to 1.5 and preferably 1.0 or less according to the present invention is physically chemically superior to calcium silicate hydrate (C—S—H) having a calcium/silicon molar ratio of 1.0 to 2.0, produced from a conventional medical filler composition including Portland cement.

As the calcium/silicon molar ratio decreases, the average length of a silicate chain and the interlayer distance of C—S—H may increase, and thus the calcium silicate hydrate (C—S—H) according to the present invention has a large specific surface area (Brunauer-Emmett-Teller, BET) compared to the conventional calcium silicate hydrate (C—S—H) of Portland cement.

When the calcium/silicon molar ratio of calcium silicate hydrate (C—S—H) is 1.0 or less, biocompatibility may drastically increase, and the calcium silicate hydrate (C—S—H) according to the present invention has superior bioactivity and biocompatibility.

Meanwhile, water may be present in various forms, such as capillary water present in a space larger than 5 nm, absorbed water binding to the surface of hydrate particles through hydrogen bonding, interlayer water, and the like, inside the calcium silicate hydrate (C—S—H) produced from the medical filler composition of the present invention. Thereby, it is possible to form a water environment close to the natural state even after endodontic obturation treatment, unlike the conventional medical filler composition. Furthermore, the risk of root fracture due to the endodontic treatment may be decreased.

Also, calcium silicate hydrate (C—S—H) produced from the medical filler composition of the present invention is a neutral compound that is biochemically stable, and may be particularly effectively used for perforation repair of a portion that contacts the saliva or gingival crevicular fluid because it is not corroded by the saliva or gingival crevicular fluid, but is not limited thereto, and may be effectively used for root canal filling of the permanent tooth and deciduous tooth, root perforation repair, pulpotomy, partial pulpotomy, pulp capping, root-end retrofilling, and the like.

Furthermore, a conventional medical filler composition is susceptible to micro-leakage, so after much hard treatment, many portions of the tooth have to be deleted and a metal crown made of stainless steel has to be fitted to provide an additional seal, but the medical filler composition of the present invention is advantageous in that it is free from micro-leakage because of its excellent sealing ability. Thus, even when part of the pulp floor is merely coated with the medical filler composition of the present invention after the endodontic obturation treatment, secondary infection may be suppressed.

In the present invention, the calcium supply source is in a powder phase, and preferably has a specific surface area (BET) having a size sufficient to produce calcium silicate hydrate (C—S—H) through an efficient pozzolanic reaction with the silicon supply source. To this end, a nanoparticle size of 100 nm or less is preferable.

In the present invention, the silicon supply source is also in a powder phase, and preferably has a specific surface area (BET) having a size sufficient to produce calcium silicate hydrate (C—S—H) through an efficient pozzolanic reaction with the calcium supply source, and preferably a specific surface area (BET) of 100 m²/g or more.

Furthermore, when the silicon supply source having a large specific surface area (BET) is used in this way, a shear thickening effect may occur, whereby the medical filler composition of the present invention undergoes gelation while pressure is drastically increased in the narrow space of the root end. Accordingly, upon root canal filling, the medical filler composition of the invention does not overflow the root end even when injected with excessive pressure over a predetermined magnitude, so it is excellent in terms of safety.

Thus, the medical filler composition of the invention may be particularly effectively applied to deciduous teeth, in which the means by which the root canal length may be adjusted has depended only on the skill of practitioners, unlike the permanent teeth of adults. It is possible to solve the problem in which the medical filler composition overflows not only the root end but also the permanent tooth bud due to excessive injection pressure.

The medical filler composition of the present invention is preferably provided in a paste form so as to be easily injected into a space requiring in-vivo repair and filling and so as to be easily stored. To this end, a liquid material is preferably included therein. Specifically, the medical filler composition of the present invention is provided in a paste form by mixing and kneading the calcium supply source and the silicon supply source with the liquid material.

The liquid material is preferably used in an amount of 20 to 70 parts by weight based on 100 parts by weight of a mixture comprising the calcium supply source and the silicon supply source. If the amount thereof is less than 20 parts by weight, it is difficult to perform mixing and kneading. On the other hand, if the amount thereof exceeds 70 parts by weight, the medical filler composition may become excessively dilute, making it difficult to inject or store.

The liquid material is preferably a liquid that is polar, less viscous and easily miscible with water and has superior penetration-enhancing properties and may be safely used in the human body, and may include at least one selected from among N-methyl-2-pyrrolidone (NMP), dimethyl sulfoxide (DMSO) and diethylene glycol monoethyl ether (DEGEE).

The medical filler composition of the present invention, formed into a paste through the addition of the liquid material, may be gradually absorbed into the surrounding tissue even after injection into a space requiring in-vivo repair and filling. When water is introduced from the surrounding tissue, the calcium supply source and the silicon supply source are subjected to a pozzolanic reaction, thereby producing calcium silicate hydrate (C—S—H).

The medical filler composition of the present invention preferably has radiopacity so that the degree of progress of the procedure may be accurately determined through observation using radiation transmission. To this end, a radiopaque powder is preferably further included.

The radiopaque powder preferably includes at least one selected from among a ferroelectric material, bismuth oxide, zirconium oxide, tantalum pentoxide, bismuth subnitrate, calcium tungstate and barium sulfate. Particularly useful is a ferroelectric material.

The ferroelectric material is a material that has the characteristic of being capable of changing the direction of polarization thereof by an external electric field because it is spontaneously polarized without an external electric field. Preferably useful is a perovskite-type metal oxide powder including at least one selected from among bismuth and barium, and specifically, at least one selected from among bismuth titanate (Bi₄Ti₃O₁₂) powder and barium titanate (BaTiO₃) powder.

The ferroelectric material is able to generate a bioelectrical signal with electrical characteristics such as piezoelectric characteristics, and not only promotes cell growth, but also has radiopaque properties, low cytotoxicity, and excellent biocompatibility and chemical resistance.

Thus, the medical filler composition of the present invention, including the ferroelectric material powder as the radiopaque powder, may exhibit radiopacity, low cytotoxicity, and high biocompatibility and chemical resistance, and may also promote cell growth when loaded into a space formed by removing nerves, blood vessels, cell tissues and hard tissues in vivo.

The radiopaque powder is preferably contained in an amount suitable for the purpose of use of the medical filler composition of the present invention and the kind of radiopaque powder.

More specifically, the radiopaque powder may be used in an amount of 20 to 300 parts by weight based on 100 parts by weight of the mixture comprising the calcium supply source and the silicon supply source. The radiopaque powder may be contained in a relatively small amount upon perforation repair, partial pulpotomy or pulp capping, or may be contained in a relatively large amount when used as a sealer for root canal filling.

When the radiopaque powder is bismuth titanate (Bi₄Ti₃O₁₂) powder, it is preferably used in an amount of 20 to 35 parts by weight based on 100 parts by weight of the mixture comprising the calcium supply source and the silicon supply source. When the radiopaque powder is a barium titanate (BaTiO₃) powder, it is preferably used in an amount of 40 to 300 parts by weight based on 100 parts by weight of the mixture comprising the calcium supply source and the silicon supply source.

The radiopaque powder is preferably a powder, the surface of which is coated with silica (SiO₂). This is because a silica coating layer suppresses leaching of the radiopaque powder to thus prevent discoloration of the tooth, thereby increasing the aesthetic appearance thereof and further increasing biocompatibility.

The silica coating layer induces a pozzolanic reaction, whereby the structure of the cured medical filler composition, obtained by curing the medical filler composition of the present invention, becomes denser, thus increasing workability and sealing ability.

The medical filler composition of the present invention may further include a smectite clay mineral in order to require expansion while curing or to increase antimicrobial effects.

The smectite clay mineral is preferably a material that is merely related to the expansion of the medical filler composition without causing a pozzolanic reaction with the calcium supply source, unlike a smectite clay mineral usable as the silicon supply source.

The smectite clay mineral may include at least one selected from among bentonite and hectorite, and is preferably contained so as to have a volume expanded by 1 to 3%, and preferably about 2%, of the volume before curing, after curing of the medical filler composition of the present invention.

The volume expansion rate is set in the optimal range so as to prevent cracking of the cured medical filler composition while a space that could not be completely filled in the conventional art, even when meticulously filled by a practitioner, is thoroughly filled with the medical filler composition of the invention.

The medical filler composition of the present invention may further include calcium aluminate and calcium sulfate to promote curing so as to ensure rapid condensation properties.

The calcium aluminate preferably includes at least one selected from among tricalcium aluminate (C₃A) and dodecacalcium hepta-aluminate (C₁₂A₇), and may be used in an amount of 15 parts by weight or less based on 100 parts by weight of the mixture comprising the calcium supply source and the silicon supply source.

The calcium sulfate may be used in an amount of 50 parts by weight based on 100 parts by weight of the calcium aluminate, but is not particularly limited thereto, and may be contained in an amount of 100 parts by weight or less based on 100 parts by weight of the calcium aluminate.

The medical filler composition of the present invention may further include a polyol in order to exhibit anti-biofilm effects and to serve as a viscosity modifier and a dispersing agent. The polyol is contained in an amount of 10 parts by weight or less, preferably 6 to 9 parts by weight, and more preferably 7 parts by weight, based on 100 parts by weight of the liquid material.

The polyol may include at least one selected from among xylitol and erythritol, which exhibit superior anti-biofilm effects and outstanding dispersion effects of strong base materials such as calcium hydroxide and calcium oxide as calcium supply sources even when used in small amounts, and may also lower the freezing point of dimethyl sulfoxide (DMSO), which is mainly used as the liquid material.

For example, dimethyl sulfoxide (DMSO) is a safe chemical with little toxicity to the human body, but it has a high freezing point of 18.5° C., which makes it inconvenient to use in cold weather. In this case, erythritol is added in an amount of 5 to 10 parts by weight based on 100 parts by weight of DMSO, whereby the freezing point of DMSO may be lowered to 4° C. or less.

In addition to the direct injection into a space requiring in-vivo repair and filling, the medical filler composition of the present invention may be placed in a container such as a vial in which water is present and may thus be rotated using a device that rotates at high speed, thereby producing and using calcium silicate hydrate (C—S—H) having a very small size.

As described above, the medical filler composition of the present invention may be employed in dental applications such as pulp capping, pulpotomy, retrofilling, perforation repair and root canal filling, and thus the amounts of the components of the medical filler composition of the present invention may be adjusted to have suitable compressive strength for such various purposes of use.

Generally, high compressive strength is favorable. In order for the medical filler composition of the invention to serve as a root canal filler for a deciduous tooth, which should respond to the eruption of a permanent tooth by being removed by the permanent tooth, and to increase the convenience of re-treatment, compressive strength may be controlled to be 15 MPa or less.

The medical filler composition of the present invention, which is injected into a space requiring in-vivo repair and filling, may be sonicated during curing and thus foaming thereof may be minimized.

For example, when the medical filler composition of the present invention is used as a root canal filler, the medical filler composition is injected into the middle third of the root canal of a permanent tooth, and is then pushed into the root canal to the correct length using a gutta percha cone. Thereafter, when the gutta percha cone is subjected to ultrasonic vibration, foam inside the root canal escapes upwardly by ultrasonic vibration, consequently preventing foaming in the medical filler composition.

Below is a detailed description of the present invention through the following Preparation Examples, Comparative Examples and Examples. All the reagents used herein are those which are generally commercially available, and they are used without special purification, unless otherwise stated. Furthermore, the following Preparation Examples, Comparative Examples and Examples are set forth to illustrate but are not to be construed as limiting the scope of the present invention.

### PREPARATION EXAMPLE 1

Respective medical filler compositions were prepared by mixing calcium hydroxide (Ca(OH)₂), precipitated silica and dimethyl sulfoxide (DMSO) so as to produce calcium silicate hydrates (C—S—H) at calcium/silicon molar ratios of 1.5, 1.2, 0.9 and 0.7 through a pozzolanic reaction, as shown in Table 1 below. Each of the medical filler compositions was then exposed at a humidity of 100% and a temperature of 36° C. to thus complete curing thereof, thereby obtaining cured medical filler compositions.

TABLE 1

| Prep. Example | Calcium (Ca)/silicon (Si) molar ratio |
|---|---|
| 1-1 | 1.5 |
| 1-2 | 1.2 |
| 1-3 | 0.9 |
| 1-4 | 0.7 |

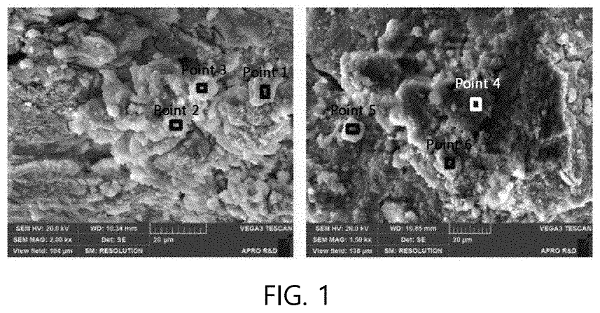

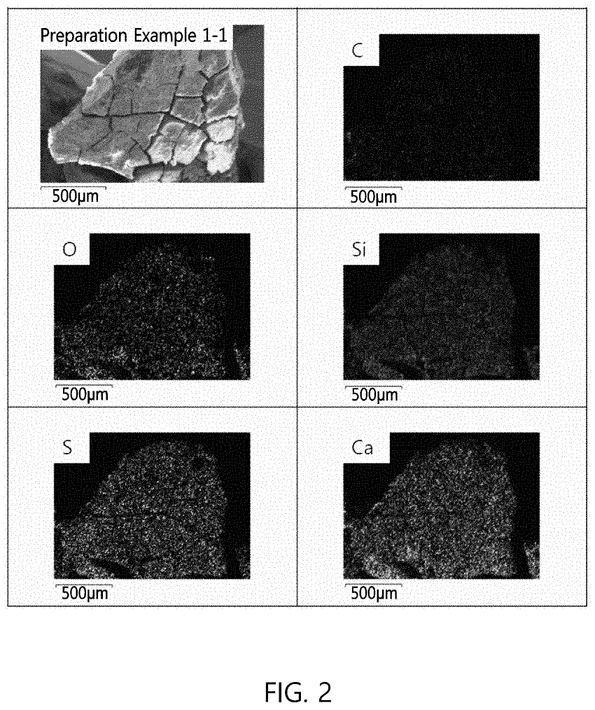

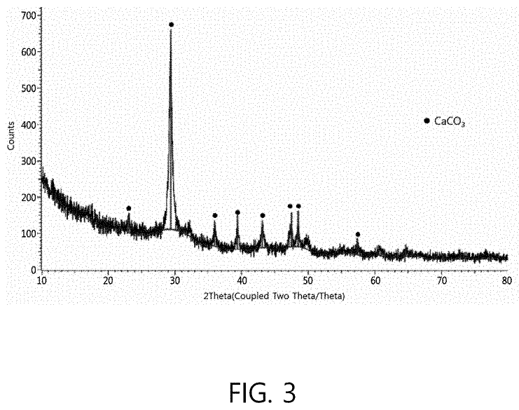

FIG. 1 shows the image results observed by FE-SEM of the cured medical filler composition of Preparation Example 1-1, FIG. 2 shows the results of coverage and distribution through EDS mapping, and FIG. 3 is a graph showing the results of XRD. Table 2 below shows the analysis results of the presence of elements through EDS.

TABLE 2

| Analytical | | | | | |
|---|---|---|---|---|---|
| sample | C[wt %] | O[wt %] | Si[wt %] | S[wt %] | Ca[wt %] |
| Point 1 | 18.84 | 42.19 | 11.59 | 8.80 | 18.88 |
| Point 2 | 24.25 | 50.34 | 8.48 | 6.81 | 10.12 |
| Point 3 | 16.87 | 38.61 | 13.74 | 8.27 | 22.50 |
| Point 4 | 29.97 | 43.67 | 8.77 | 8.38 | 9.21 |
| Point 5 | 27.93 | 50.71 | 7.80 | 5.93 | 7.63 |
| Point 6 | 30.22 | 49.85 | 6.23 | 7.24 | 6.46 |

As is apparent from Table 2 and FIGS. 1 to 3, calcium carbonate (CaCO₃) was produced in the cured medical filler composition of Preparation Example 1-1, resulting from reacting part of calcium silicate hydrate (C—S—H) with carbon dioxide in the air. This reaction may be the same as the reaction that occurs in the human body, and thus, the sealing ability of the cured medical filler composition is considered to be further increased due to the presence of a very small amount of calcium carbonate thus produced.

Based on the results of observation of the cured medical filler compositions of Preparation Example 1, none of the cured medical filler compositions of Preparation Examples 1-1 to 1-4 underwent volume shrinkage.

TABLE 3

| Analytical sample | Length of micro-leakage Mean ± SD [mm] |
|---|---|
| AH-plus | 1.073 ± 0.9153 |
| Preparation Example 1-1 | 1.703 ± 1.0254 |
| Preparation Example 1-4 | 1.185 ± 0.9705 |

Table 3 shows the results of testing of sealing ability of the cured medical filler compositions of Preparation Examples 1-1 and 1-4 and a commercially available cured medical filler composition. The testing method thereof was as follows.

In the present testing, resin-based AH-plus was used as the conventional medical filler composition, which is a proven product most commonly used as a control in the academic world.

The medical filler composition was injected into the middle third of the root canal of a permanent tooth, and was then pushed into the root canal to the correct length using a gutta percha cone, after which the gutta percha cone was subjected to ultrasonic vibration, whereby foam inside the root canal escaped upwardly upon ultrasonic vibration. Thereafter, two coating processes were performed using a nail varnish, except for 2 mm of the radius of the apical foramen of the tooth, followed by immersion in a saline solution and then curing at 37° C. for 24 hr. Thereafter, the apical one-third of the sample was immersed in a 0.2% rhodamine B dye solution, maintained at 37° C. for 24 hr, taken out of the dye solution, and washed with water. Thereafter, the nail varnish was removed, the sample was split in a longitudinal direction, and micro-leakage was observed, and thus the length of micro-leakage ranging from the apex to the most deeply dyed portion was measured. In Table 3, Mean±SD is a mean value±standard deviation.

As shown in Table 3, none of the cured medical filler compositions of Preparation Examples 1-1 and 1-4 had statistically significant difference from AH-plus. The cured medical filler composition of the present invention was determined to exhibit very good sealing ability.

In particular, the length of the cured medical filler composition of Preparation Example 1-4 was very similar to that of AH-plus compared to Preparation Example 1-1, which means that the sealing ability of the cured medical filler composition of Preparation Example 1-4 was superior to that of the cured medical filler composition of Preparation Example 1-1. Thereby, the cured medical filler composition in which calcium silicate hydrate (C—S—H) at a calcium/silicon molar ratio of 1.0 or less was produced can be concluded to exhibit outstanding sealing ability.

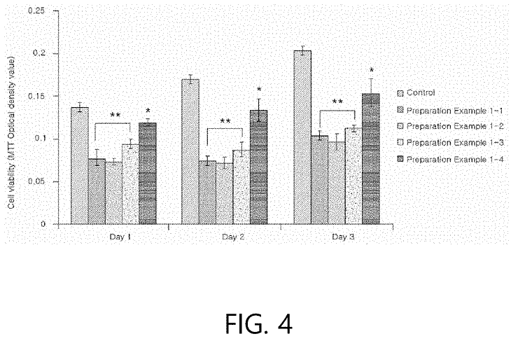

FIG. 4 is a graph showing the results of MTT analysis of cytotoxicity of the cured medical filler compositions of Preparation Example 1. The testing method thereof is as follows.

A test sample having a diameter of 10 mm and a thickness of 2 mm was manufactured and stored in an incubator at 37° C. under constant humidity for 3˜7 days. Thereafter, the test sample was exposed to UV light overnight and thus sterilized, and extracted at a concentration of 0.5 cm²/ml in a 37° C. incubator for 3 days, and the supernatant of the extracted medium was isolated and stored. The MC3T3-E1 cell line used for the cytotoxicity test was incubated in an MEM-a medium containing 10% FBS, and the MT3T3-E1 cell line was aliquoted into a 24-well plate at 1.5×10⁴ per well and incubated for one day. Here, the sample was prepared in 4-fold dilutions, and respective plates at days 1, 2, and 3 were prepared. Thereafter, the cultured cell line broth was removed, and the extracted medium was aliquoted in an amount of 1 ml per well and incubated, and an MTT assay was performed at days 1, 2, and 3. Specifically, the cell broth was removed, and a 0.05% MTT solution in PBS was added in an amount of 200 μl each and then incubated in a 37° C. incubator for 2 hr. Thereafter, a DMSO solution was added in an amount of 200 μl each. After 10 min, 200 μl was removed from each well of the 96-well plate, and optical density (OD) was measured to thus evaluate the cell survival rate. Here, the cell survival rate was determined using the mean and standard deviation of the measurement results of the three test groups.

As shown in FIG. 4, the cell survival rate of the cured medical filler composition of Preparation Example 1-1 was very similar to that of the cured medical filler composition of Preparation Example 1-2. In contrast, the cured medical filler compositions of Preparation Examples 1-3 and 1-4 exhibited high cell survival rates. In particular, the cured medical filler composition of Preparation Example 1-4 showed a statistically significant difference and thus a remarkably high cell survival rate.

Hence, the cured medical filler composition in which calcium silicate hydrate (C—S—H) at a calcium/silicon molar ratio of 1.0 or less was produced exhibited relatively low cytotoxicity and high bioactivity.

### EXAMPLE 1

A cured medical filler composition was manufactured in the same manner as in Preparation Example 1-1, with the exception that when mixing calcium hydroxide (Ca(OH)₂), precipitated silica and dimethyl sulfoxide (DMSO), 5 parts by weight of calcium aluminate and 5 parts by weight of calcium sulfate were further added based on 100 parts by weight of a mixture comprising calcium hydroxide and precipitated silica.

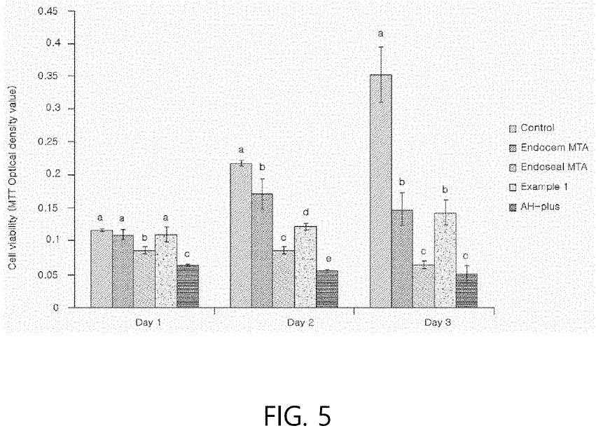

FIG. 5 is a graph showing the results of testing through MTT analysis for cytotoxicity of the cured medical filler composition of Example 1 and commercially available cured medical filler compositions, and the testing method thereof is as described above.

The conventional medical filler compositions used in this test were Endocem MTA, Endoseal MTA and AH-plus, and more specifically, Endocem MTA mixing with water without the additional use of a liquid material, calcium silicate-based Endoseal MTA using N-methyl-2-pyrrolidone (NMP) as a liquid material, and resin-based AH-plus, commonly serving as a control in the academic world.

As shown in FIG. 5, the cell survival rate of the cured medical filler composition of Example 1 was high on all of days 1, 2 and 3 compared to Endoseal MTA and AH-plus. Thus, the cured medical filler composition of the present invention can be found to exhibit low cytotoxicity and high bioactivity compared to Endoseal MTA and AH-plus.

Moreover, the cell survival rate of the cured medical filler composition of Example 1 had no statistically significant difference except for day 2, compared to Endocem MTA, from which the cured medical filler composition of the present invention was determined to exhibit very good cytotoxicity and bioactivity.

### EXAMPLE 2

A cured medical filler composition was manufactured in the same manner as in Preparation Example 1-1, with the exception that when mixing calcium hydroxide (Ca(OH)₂), precipitated silica and dimethyl sulfoxide (DMSO), 200 parts by weight of barium titanate (BaTiO₃) was further added based on 100 parts by weight of a mixture comprising calcium hydroxide and precipitated silica.

### EXAMPLE 3

A cured medical filler composition was manufactured in the same manner as in Preparation Example 1-1, with the exception that when mixing calcium hydroxide (Ca(OH)₂), precipitated silica and dimethyl sulfoxide (DMSO), 5 parts by weight of calcium aluminate based on 100 parts by weight of a mixture comprising calcium hydroxide and precipitated silica and 70 parts by weight of calcium sulfate anhydride based on 100 parts by weight of calcium aluminate were further added. Here, the calcium aluminate was a mixture comprising tricalcium aluminate (C₃A) and dodecacalcium hepta-aluminate (C₁₂A₇) mixed at a weight ratio of 3:7.

The cured medical filler compositions of Examples 2 and 3 can also be concluded to exhibit very good cytotoxicity and bioactivity.

Although the preferred embodiments of the present invention regarding the medical filler composition of the present invention have been disclosed for illustrative purposes, they are not limited to the aforementioned examples and the appended drawings and thus are not to be construed as limiting the scope of the present invention. Therefore, the technical scope of the present invention is to be determined by the technical ideas of the accompanying claims. Moreover, those skilled in the art will appreciate that various modifications, additions and substitutions are possible, without departing from the scope and spirit of the invention as disclosed in the accompanying claims.

### INDUSTRIAL APPLICABILITY

The present invention relates to a medical filler composition, and more particularly to a medical filler composition, which is loaded in a space that is empty due to the removal of nerves, blood vessels, soft tissues and hard tissues therefrom.

According to the present invention, a medical filler composition can produce calcium silicate hydrate (C—S—H) through a pozzolanic reaction after injection into an environment in which water is present, namely a space requiring in-vivo repair and filling. A cured medical filler composition containing the calcium silicate hydrate (C—S—H) thus produced is a neutral compound that is biocompatible and has high bioactivity and high biochemical stability, and can be effectively used for perforation repair of a portion that contacts the saliva or gingival crevicular fluid because it is not corroded by the saliva or gingival crevicular fluid.

Furthermore, the medical filler composition of the invention can exhibit high workability and sealing ability and is thus free from micro-leakage, thus obviating an additional operation for sealing and inhibiting secondary infection. Also, the medical filler composition of the invention does not overflow the root end even when injected with excessive pressure over a predetermined magnitude, and is thus excellent in terms of safety.

Moreover, the medical filler composition of the invention is in a paste form and is thus easily injected into a space requiring in-vivo repair and filling and can be effectively absorbed to the surrounding tissue even after injection.

1. A medical filler composition, comprising:
a calcium supply source including at least one selected from among calcium hydroxide and calcium oxide;
a silicon supply source including at least one selected from fumed silica, precipitated silica, colloidal silica and clay mineral; and
20 to 70 parts by weight of a liquid material for pasting, based on 100 parts by weight of a mixture comprising the calcium supply source and the silicon supply source,
wherein a calcium/silicon molar ratio of a calcium silicate hydrate produced by a pozzolanic reaction between the calcium supply source and the silicon supply source is 0.25 to 1.5.

2. The medical filler composition of claim 1, wherein the calcium supply source has a particle size of 100 nm or less.

3. The medical filler composition of claim 1, wherein the silicon supply source has a specific surface area of 100 m²/g or more.

4. The medical filler composition of claim 1, wherein the liquid material includes at least one selected from among N-methyl-2-pyrrolidone (NMP), dimethyl sulfoxide (DMSO) and diethylene glycol monoethyl ether (DEGEE).

5. The medical filler composition of claim 1, further comprising a radiopaque powder.

6. The medical filler composition of claim 5, wherein the radiopaque powder includes at least one selected from among a ferroelectric material, bismuth oxide, zirconium oxide, tantalum pentoxide, bismuth subnitrate, calcium tungstate and barium sulfate.

7. The medical filler composition of claim 6, wherein the ferroelectric material is a perovskite-type metal oxide including at least one selected from among bismuth titanate (Bi₄Ti₃O₁₂) and barium titanate (BaTiO₃).

8. The medical filler composition of claim 5, wherein the radiopaque powder is coated with silica.

9. The medical filler composition of claim 1, further comprising a smectite clay mineral so as to have a volume expanded by 1 to 3% of a volume before curing, after curing of the medical filler composition.

10. The medical filler composition of claim 9, wherein the smectite clay mineral includes at least one selected from among bentonite and hectorite.

11. The medical filler composition of claim 1, further comprising a polyol in an amount of 10 parts by weight or less based on 100 parts by weight of the liquid material.

12. The medical filler composition of claim 11, wherein the polyol includes at least one selected from among xylitol and erythritol.

13. The medical filler composition of claim 1, further comprising calcium aluminate and calcium sulfate.

14. The medical filler composition of claim 13, wherein the calcium aluminate includes at least one selected from among tricalcium aluminate (C₃A) and dodecacalcium hepta-aluminate (C₁₂A₇).

15. The medical filler composition of claim 1, wherein the medical filler composition is used for dental application.

16. The medical filler composition of claim 15, wherein the medical filler composition has a compressive strength of 15 MPa or less and is used as a root canal filler for a deciduous tooth.

17. The medical filler composition of claim 15, wherein the medical filler composition is minimized in foaming by application of ultrasonic waves from outside during curing thereof.
